(19) United States
(12) Patent Application Publication
(10) Pub. No.: US 2015/0094728 A1
(43) Pub. Date: Apr. 2, 2015

(54) INSTRUMENT FOR POSITIONING A CUP COMPONENT OF AN ORTHOPAEDIC JOINT PROSTHESIS

(71) Applicant: DEPUY SYNTHES PRODUCTS, LLC, Raynham, MA (US)

(72) Inventors: JOEL C. RHOADES, Pierceton, IN (US); MATTHEW D. SCHMIT, Fort Wayne, IN (US)

(21) Appl. No.: 14/040,821

(22) Filed: Sep. 30, 2013

(51) Int. Cl. A61F 2/46

(52) U.S. Cl. CPC A61F 2/4609
USPC 606/91

(57) ABSTRACT

An instrument for positioning a hollow cup component of an orthopaedic joint prosthesis. The instrument includes a longitudinal member having a first end and a second end opposite the first end. The instrument further includes a resilient cup engaging member coupled to the first end. The resilient cup engaging member has a convex outer surface adapted to engage an interior of the hollow cup component. The entire outer convex surface is manufactured of a resilient material. An actuator in fluid communication with the resilient cup engaging member is also included. When the actuator is actuated, the suction force between the resilient cup engaging member and the hollow cup increases.

### TECHNICAL FIELD

This invention relates to an instrument for positioning a cup component of an orthopaedic joint prosthesis.

### BACKGROUND

Certain orthopaedic joint prostheses include a hollow cup with an inner surface which defines a generally hemispherical hollow region, and another component which has a spherical part which can be received in the hollow region for articulation relative to the cup component. Such joint prostheses can include hip joint prostheses and shoulder joint prostheses. The exterior of the cup will contact the prepared surface of the patient's bone in which the component is to be implanted. The interior of the cup will present a smooth bearing surface to the spherical convex part of the other component of the joint prosthesis. The bearing surface can be provided by a single piece cup component. Alternatively, the cup component can comprise a shell part which contacts the prepared surface of the patient's bone, and a bearing part which provides the bearing surface, and which fits into the shell part. The bearing part can be made from a material which is different from the material of the shell part: for example the bearing part can be made from a polymeric material (such as polyethylene) and the shell part (and the spherical convex part of the other component) can be made from a metal (such as a cobalt-chromium based alloy, or a stainless steel, or a titanium based alloy).

It is important that the components of an orthopaedic joint prosthesis are positioned accurately in a patient's bone. Both location and alignment are important. Accurate positioning of a component requires that the component be engaged by an appropriate instrument, allowing considerable force to be applied to the component if and as necessary (for example through use of an impactor instrument). However, it can be important not to contact the external surface or the internal surface or both of the component with the instrument, especially the internal surface when it has been provided with a smooth polished bearing surface. Scratching or otherwise damaging that surface can impair the bearing properties of the prosthesis.

In some prior art instruments, the insertion tool includes a flange that interacts with a groove cut into an inner surface of the cup. Other prior art instruments utilize a plurality of jaw members which extend radially from a central drive shaft. The jaw members can be made to slide radially inwardly so that they engage the outside wall of a cup component. Each of the jaw members has a pin at one end which is received in a spiral track on a drive plate. The jaw members are made to slide radially by rotating the drive plate. The jaw members engage a corresponding recess in the cup.

In each of these prior art examples, the insertion tool works by interacting with special features on the cup. This limits the instrument to only be able to be used with certain cups. Therefore, there is a need for an insertion instrument that can be used on a cup without the cup having special features added to the cup.

### SUMMARY

According to one embodiment of the present invention, an instrument for positioning a hollow cup component of an orthopaedic joint prosthesis is provided. The instrument includes a longitudinal member having a first end and a second end opposite the first end. The instrument further includes a resilient cup engaging member coupled to the first end. The resilient cup engaging member has a convex outer surface adapted to engage an interior of the hollow cup component. The entire outer convex surface is manufactured of a resilient material. An actuator in fluid communication with the resilient cup engaging member is also included. When the actuator is actuated, the suction force between the resilient cup engaging member and the hollow cup increases.

According to another embodiment, a method for using an insertion instrument to insert a cup into an acetabulum is provided. The method includes inserting a convex outer surface of the instrument into an inner surface of the cup. The entire convex outer surface is made of a resilient material. A piston is activated, in order to decrease pressure within a sealed cavity of the instrument, This activation causes the convex outer surface of the instrument to engage the inner surface of the cup. The instrument and cup are inserted into place in the acetabulum. The piston is de-activated in order to increase pressure within the sealed cavity, causing the convex outer surface of the instrument to disengage the inner surface of the cup.

According to yet another embodiment of the present invention, a kit for use in hip arthroplasty is provided. The kit includes a monoblock cup, and an instrument for positioning the cup. The instrument includes a longitudinal member having a first end and a second end opposite the first end. Also included is a resilient cup engaging member coupled to the first end. The resilient cup engaging member having a convex outer surface adapted to engage an interior of the cup. The entire outer convex surface is manufactured of a resilient material. The instrument also includes an actuator in fluid communication with the resilient cup engaging member, such that when the actuator is actuated, the suction force between the resilient cup engaging member and the cup increases.

### BRIEF DESCRIPTION OF THE DRAWINGS

FIG. 1 is a perspective view of a cup inserting instrument according to one embodiment of the present invention.

FIG. 2 is a cross-sectional side view of the instrument of FIG. 1.

FIG. 3 is a perspective view of the instrument of FIG. 1 with a cup.

FIG. 4 is a perspective view of a cup according to one embodiment of the present invention.

FIG. 5 is a flow chart illustrating a method of using the instrument according to one embodiment of the present invention.

### DETAILED DESCRIPTION OF THE DRAWINGS

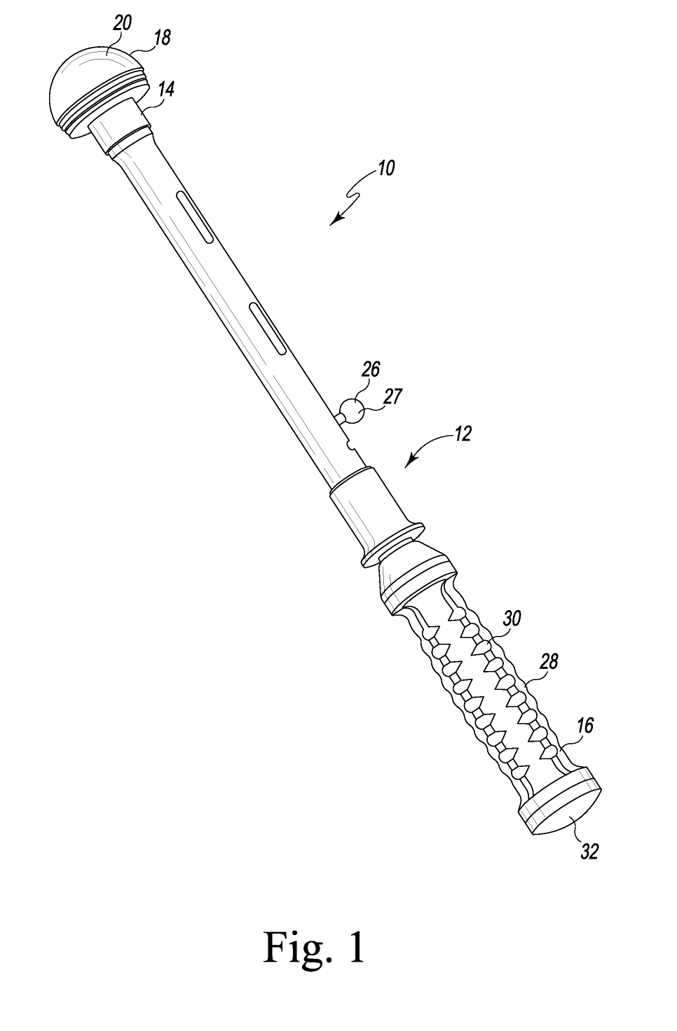

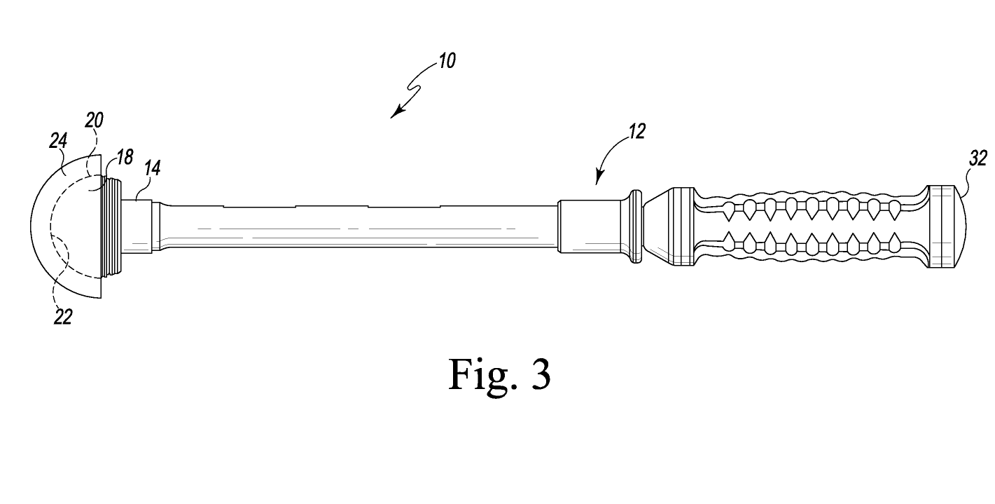

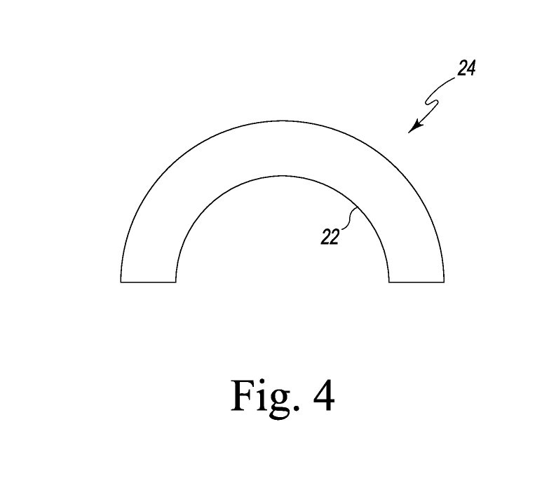

Like reference numerals refer to like parts throughout the following description and the accompanying drawings. Turning now to FIG. 1, a perspective view of a cup inserting instrument 10 is shown. The instrument includes a longitudinal member 12 having a first end 14 and a second end 16 opposite the first end 14. A resilient cup engaging member 18 is coupled to the first end 14. The resilient cup engaging member 18 includes a convex outer surface 20. The convex outer surface 20 is sized and shaped to engage an inner surface 22 of an acetabular cup 24 (see FIGS. 3 and 4). The convex outer surface 20 is made entirely of a resilient material. In some embodiments, that resilient material is medical grade silicone. In other embodiments, the resilient material may be thermoplastic elastomer (TPE) or thermoplastic urethane (TPU). Alternatively, any type of resilient material may be used.

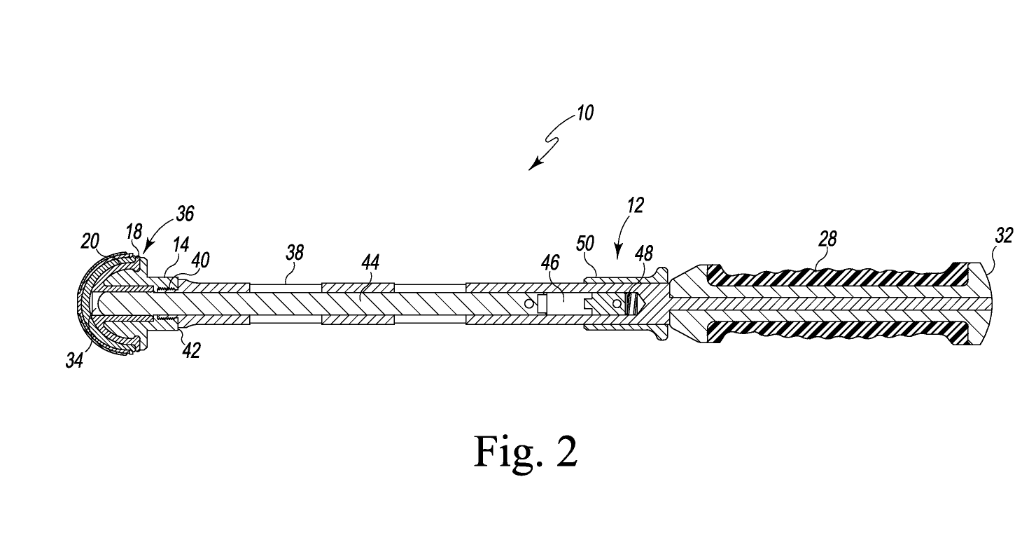

As shown in FIG. 1, the longitudinal member 12 is coupled to a lever 26, which is part of an actuator 27. The actuator 27 is in fluid communication with the resilient cup engaging member 18 such that when the actuator 27 is moved, the suction force between the resilent cup engaging member 18 and a cup 24 (shown in FIG. 4) increases. The actuator 27 will be described in further detail in reference to FIG. 2. At the second end 16 of the longitudinal member 12, a handle 28 may be included. The handle 28 may include ergonomic features such as ridges 30 to making gripping the handle 28 easier. Also, in some embodiments, the handle 28 may include an impaction plate 32 on its end. In such an embodiment, a user may strike the instrument 10 with a hammer or other impaction tool. In other embodiments, the end of the handle 28 may not be an impaction plate.

Turning now to FIG. 2, the internal workings of the cup inserting instrument 10 will be described. As shown in FIG. 2, the resilient member 18 extends over the entirety of the convex outer surface 20. It also extends inwardly to create a seal around an inner cavity 34. In some embodiments, the resilient member 18 may only extend over the convex outer surface 20 and an o-ring or separate resilient member may be used to create the seal around the inner cavity 34. As shown, the resilient member 18 is part of a housing or solid impact cap 36. The resilient member 18 may be molded over the solid impact cap or housing 36. The housing 36 includes a part of the inner cavity 34 and is made of solid metal or hard plastic in order to support the resilient member 18. In this case, the housing 36 is removable from a shaft portion 38. The housing 36 includes a threaded connection 40 that engages with a threaded connection 42 on an end of the shaft portion 38. In such an embodiment, there may be a plurality of housings 36, having resilient members 18 that correspond to different size cups 24. In other embodiments, the instrument 10 may be of a unitary structure and there may be different instruments 10 to accommodate the different size cups 24.

The shaft portion 38 is an elongated shaft and includes the remaining portion of the inner cavity 34. Inside the inner cavity 34 is also part of the actuator 27, a moveable piston 44. The moveable piston is coupled to the lever 26. As the lever 26 is moved, the moveable piston 44 moves along the inner cavity 34.

Moving toward the second end 16 of the longitudinal member 12 and shaft portion 38, is a holder 46. The holder 46 is adapted to engage the moveable piston 44 and lock the moveable piston 44 in position. There is also a spring 48, which interacts with the holder 46 and a releasement mechanism (in the illustrated embodiment, a collar) 50 in order to enable a user to unlock the moveable piston 44 from its locked position.

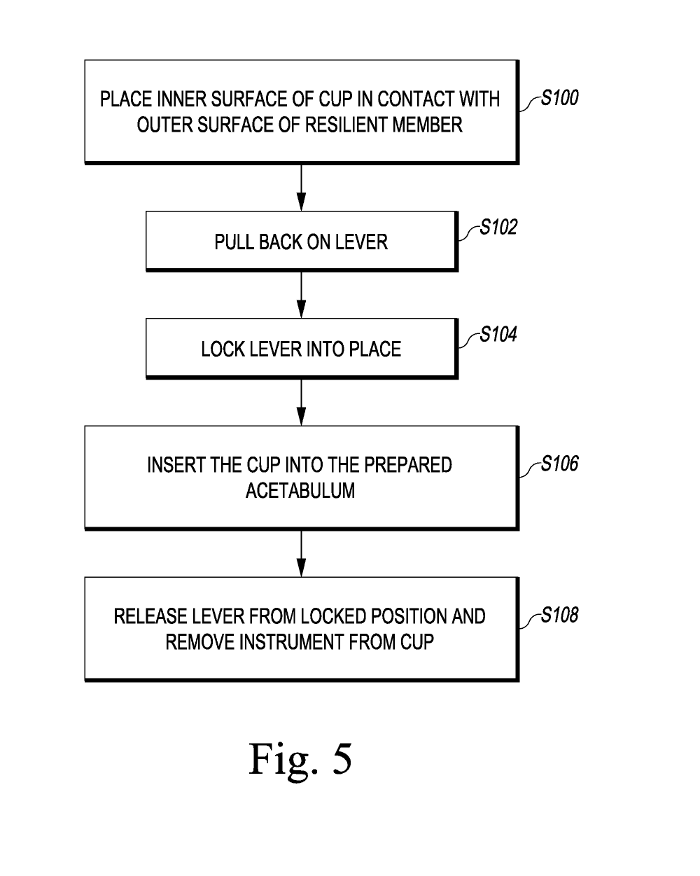

Turning now to FIG. 5, a description of how the instrument 10 will be used is described. Beginning at step s100, the inner surface 22 of the cup (also known as a shell) 24 is placed in contact with the convex outer surface 20 of the resilient member 18. The user then pulls back on the lever 26 and locks it into place at steps s102 and s104. The locking may be done by an L-shaped slot (not shown) in the longitudinal member 12. Internally, the pulling back of the lever 26 causes the moveable piston 44 to move toward the holder 46. Locking the lever 26 into the L-shaped slot, causes the moveable piston 44 to lockably engage the holder 46 (step s104). As the moveable piston 44 moves toward the second end 16, the area of air in the inner cavity 34 increases. However, since the inner cavity 34 is sealed (by either the resilient member 18 or an o-ring), the volume of air stays the same. This results in the pressure in the inner cavity 34 decreasing relative to the atmospheric pressure outside the inner cavity 34, causing the convex outer surface 20 of the resilient member to engage the inner surface 22 of the cup 24. At step 106, the user may then use the inserter 10 to insert the cup (or shell) 24 into the prepared acetabulum. This step may involve using a hammer, mallet or other tool to strike the tool to firmly seat the cup 24 in the acetabulum. Once the cup 24 is seated, the user then pulls back on the collar 50, releasing the lever 26 from its locked position (step s108). The moveable piston 44 then moves toward the first end 14 of the longitudinal member 12, increasing the pressure and releasing the cup 24 from the resilient member 18.

The cup 24 described in the above embodiments is a monoblock cup 24 having a metal outer surface and a polyethylene inner surface. The outer surface may be made of any biocompatible metal, such as titanium. The outer surface may also include a porous outer coating to promote bone in-growth. The inner surface may be molded into the outer surface and may be any biocompatible polyethylene, such as an antioxidant polyethylene. Alternatively, an ultra high molecular weight polyethylene may also be used.

The foregoing description of the invention is illustrative only, and is not intended to limit the scope of the invention to the precise terms set forth. Further, although the invention has been described in detail with reference to certain illustrative embodiments, variations and modifications exist within the scope and spirit of the invention as described and defined in the following claims.

1. An instrument for positioning a hollow cup component of an orthopaedic joint prosthesis, which comprises:
a longitudinal member having a first end and a second end opposite the first end;
a resilient cup engaging member coupled to the first end, the resilient cup engaging member having a convex outer surface adapted to engage an interior of the hollow cup component, wherein the entire outer convex surface is manufactured of a resilient material;
an actuator in fluid communication with the resilient cup engaging member, such that when the actuator is actuated, the suction force between the resilient cup engaging member and the hollow cup increases.

2. The instrument of claim 1, wherein the outer convex surface of the resilient cup engaging member is fitted over a solid impact cap.

3. The instrument of claim 1, wherein the outer convex surface is molded over a solid impact cap.

4. The instrument of claim 1, wherein the actuator includes a lever coupled to a moveable piston.

5. The instrument of claim 4, wherein the moveable piston is located in an inner cavity of the longitudinal member and is adapted to move along the inner cavity.

6. The instrument of claim 5, wherein the longitudinal member includes a locking mechanism for locking the lever in a locked position.

7. The instrument of claim 6, wherein the longitudinal member further includes a releasement mechanism coupled to the locking mechanism and adapted to release the lever from the locked position.

8. A method for using an insertion instrument to insert a cup into an acetabulum, the method comprising:
inserting a convex outer surface of the instrument into an inner surface of the cup, wherein the entire convex outer surface is made of a resilient material;
activating a piston of the instrument in order to decrease pressure within a sealed cavity of the instrument, causing the convex outer surface of the instrument to engage the inner surface of the cup;
insert the instrument and cup into place in the acetabulum; and
de-activate the piston of the instrument in order to increase pressure within the sealed cavity, causing the convex outer surface of the instrument to disengage the inner surface of the cup.

9. The method of claim 8, further comprising locking the piston after activation in order to keep the inner surface of the cup engaged with the convex outer surface.

10. The method of claim 9, wherein the activating includes pulling a lever on a shaft of the instrument and the locking includes inserting the lever into an l-shaped slot.

11. The method of claim 9, further comprising unlocking the piston after the insertion of the cup in the acetabulum.

12. The method of claim 11, wherein the unlocking includes pulling a collar on the shaft of the instrument to release the lever from the l-shaped slot.

13. A kit for use in hip arthroplasty, the kit comprising:
a monoblock cup; and
an instrument for positioning the cup, the instrument including a longitudinal member having a first end and a second end opposite the first end, a resilient cup engaging member coupled to the first end, the resilient cup engaging member having a convex outer surface adapted to engage an interior of the cup, wherein the entire outer convex surface is manufactured of a resilient material, an actuator in fluid communication with the resilient cup engaging member, such that when the actuator is actuated, the suction force between the resilient cup engaging member and the cup increases.

14. The kit of claim 13, wherein the monoblock cup includes a metal outer surface and the interior of the cup is made of polyethylene.

15. The kit of claim 13, wherein the instrument includes a housing adapted to support the resilient cup engaging member.

16. The kit of claim 15, wherein the instrument includes an o-ring adapted to seal a cavity within the housing.

17. The kit of claim 15, wherein the resilient cup engaging member is molded over a cavity in the housing in order to seal the cavity.

18. The kit of claim 13, wherein the actuator includes a lever coupled to a moveable piston.

19. The kit of claim 18, wherein the actuator further includes a locking mechanism designed to lock the moveable piston in a locked position.

20. The kit of claim 19, wherein the instrument further includes a collar coupled to the actuator to release the moveable piston from the locked position.
